(19) United States
(12) Patent Application Publication
(10) Pub. No.: US 2010/0228137 A1
(43) Pub. Date: Sep. 9, 2010

(54) HEART RATE ALARM SYSTEM AND METHOD

(75) Inventor: TING-JING GU, Shenzhen City (CN)

Correspondence Address: PCE INDUSTRY, INC.;ATT. Steven Reiss, 288 SOUTH MAYO AVENUE, CITY OF INDUSTRY, CA, 91789, US

(73) Assignees: SHENZHEN FUTAIHONG PRECISION INDUSTRY CO., LTD., ShenZhen City (CN); CHI MEI COMMUNICATION SYSTEMS, INC., Tu-Cheng City (TW)

(21) Appl. No.: 12/608,158

(22) Filed: Oct. 29, 2009

(30) Foreign Application Priority Data
Mar. 7, 2009 (CN) 200910300735.5

(51) Int. Cl. A61B 5/024

(52) U.S. Cl. 600/508

(57) ABSTRACT

An electronic device and method for alarming using an electronic device receives periodically a plurality of heart rates of a user from an accessory device using a BLUETOOTH device of the electronic device in a time duration, and compares the plurality of heart rates sequentially. The system and method further generates an alarm sound to remind the user by an alarm clock of the electronic device, in response to a determination that the plurality of heart rates has decreased gradually. Furthermore, the system and method resets the heart rates to zero, in response to a determination that the plurality of heart rates has not decreased gradually.

### BACKGROUND

1. Technical Field

Embodiments of the present disclosure are related to alarm systems and methods, and particularly to a system and method that triggers an alarm according to a heart rate of a person.

2. Description of Related Art

Since an electronic device (e.g., a mobile phone) is often used as personal organizer, it frequently offers personalization capabilities, such as the ability to have an alarm clock sound at a preset time. For example, the alarm clock may generate a sound to remind a user in the morning at a preset time (e.g., at 7:00 am). If the user drives a car at night, he/she may doze off during driving because of being tired out. It is very essential to remind the user timely if the user is in such situation.

Therefore, there is a need for a system and method to overcome the aforementioned problem.

### BRIEF DESCRIPTION OF THE DRAWINGS

FIG. 1 is a block diagram of one embodiment of an electronic device 1 comprising an alarm system.

FIG. 2 is a flowchart of one embodiment of a method for alarming using the electronic device 1, such as, for example, that of FIG. 1.

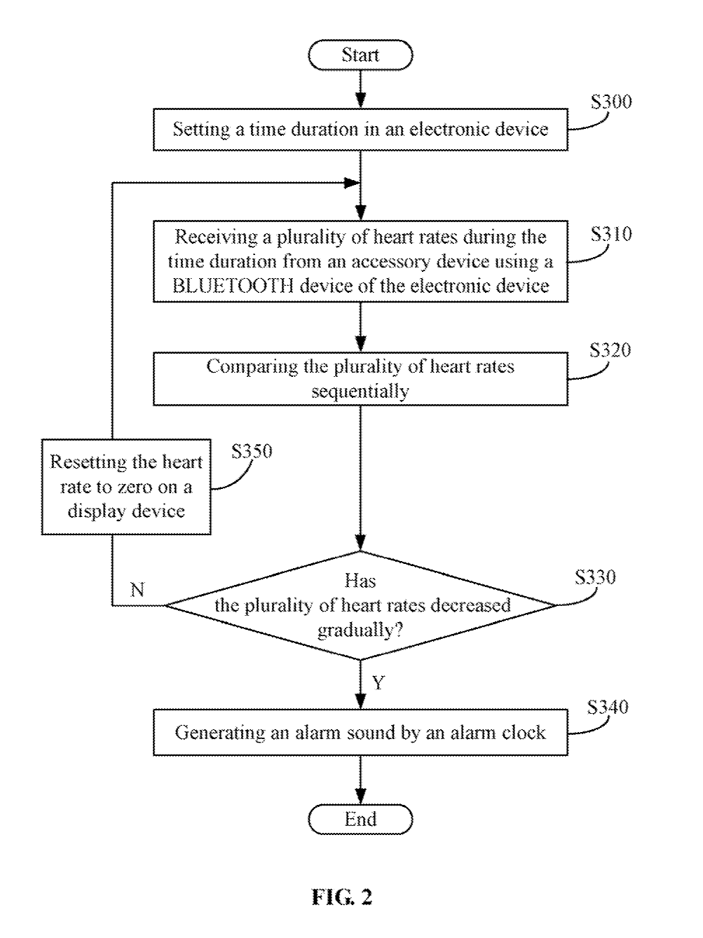

FIG. 3 illustrates one embodiment of a display interface having heart rate of a user.

FIG. 4 illustrates one embodiment of a display interface having no heart rate of the user.

### DETAILED DESCRIPTION

All of the processes described herein may be embodied in, and fully automated via, function modules executed by one or more general purpose processors of an electronic device. Some or all of the methods may alternatively be embodied in specialized hardware. The function modules may be stored in any type of computer-readable medium or other computer storage device.

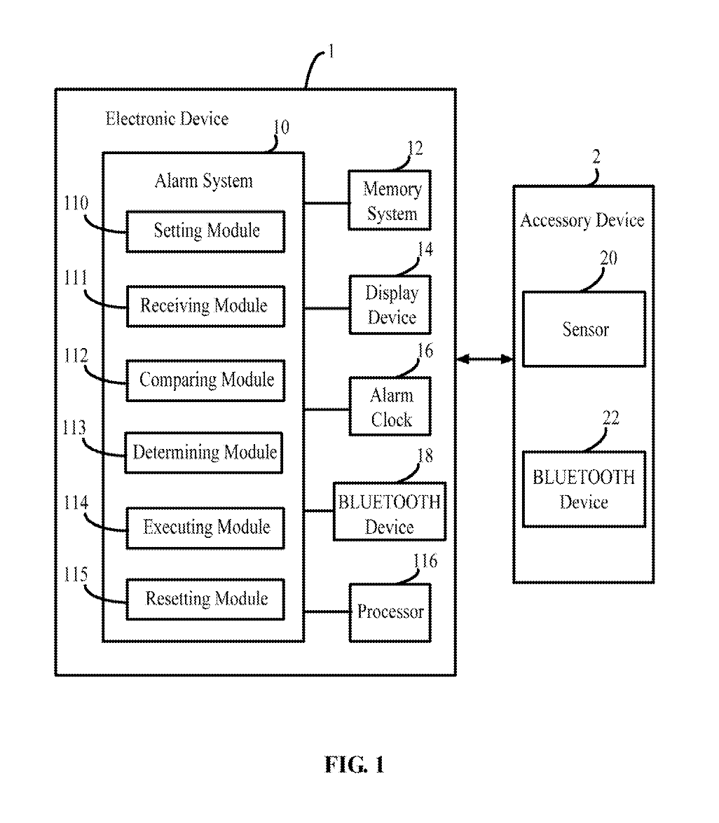

FIG. 1 is a block diagram of one embodiment of an electronic device 1 comprising an alarm system 10. The alarm system 10 may use an alarm clock 16 and a sensor 20 of the electronic device 1 to generate an alarm sound according to a heart rate of a user of the electronic device 1. In one embodiment, the electronic device 1 includes a memory system 12, a display device 14, the alarm clock 16, and a BLUETOOTH device 18. The electronic device 1 wirelessly communicates with an accessory device 2 over a BLUETOOTH connection. The accessory device 2 includes the sensor 20 and a BLUETOOTH device 22. The accessory device 2 may be attached to an arm or a wrist of the user. The accessory device 2 may be a bracelet or a chain, for example.

The memory system 12 may be an internal memory system card or an external memory system card, such as a smart media card (SMC), a secure digital card (SDC), a compact flash card (CFC), a multi media card (MMC), a memory system stick (MS), an extreme digital card (XDC), or a trans flash card (TFC). Depending on the embodiment, the electronic device 1 may be a mobile phone, a personal digital assistant (PDA), a handheld game player, a digital camera, or any other portable electronic device.

In one embodiment, the alarm system 10 includes a setting module 110, a receiving module 111, a comparing module 112, a determining module 113, an executing module 114, and a resetting module 115. One or more computerized codes of the modules 110-115 are stored in the memory system 12. One or more general purpose or specialized processors, such as a processor 116 executes the one or more computerized codes of the modules 110-115 to provide one or more operations of the electronic device 1.

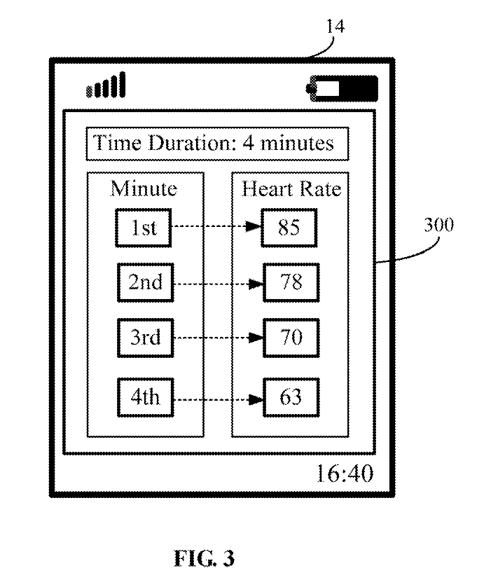

The setting module 110 sets a time duration in order to measure whether a user wearing the accessory device 2 has fallen asleep, and stores the time duration into the memory system 12 of the electronic device 1. In one embodiment, as shown in FIG. 3, for example, the setting module 110 sets the time duration as 4 minutes.

The receiving module 111 receives a plurality of heart rates of the user during the time duration from the accessory device 2 using the BLUETOOTH device 16, and displays the plurality of heart rates on the display device 14. In one embodiment, the accessory device 2 periodically (e.g., 1 minute) measures a heart rate of the user using the sensor 20 and sends the heart rate to the electronic device 1 using the BLUETOOTH device 22. For example, the accessory device 2 measures four heart rates 85 beats per minute, 78 beats per minute, 70 beats per minute, and 63 beats per minute within a 4 minutes duration. As shown in FIG. 3, the receiving module 111 receives the four heart rates 85, 78, 70, and 63 from the accessory device 2, then displays the four heart rates 85, 78, 70, and 63 on a display interface 300 of the display device 14.

The comparing module 112 compares the plurality of heart rate sequentially. In one embodiment, for example, the comparing module 112 compares the heart rate 85 with the heart rate 78, then compares the heart rate 78 with 70, and compares the heart rate 70 with the heart rate 63.

The determining module 113 determines if the plurality of heart rates has decreased gradually according to the comparison. In one embodiment, as shown in FIG. 3, heart rates are 85, 78, 70, and 63, then the plurality of heart rates has decreased gradually, for example.

In response to a determination that the plurality of heart rates has decreased gradually, the executing module 114 generates an alarm sound to remind the user using the alarm clock 14. In one embodiment, if the plurality of heart rates of the user has decreased gradually, it may indicate that the user has fallen asleep, and then the executing module 114 generates an alarm sound to remind the user using the alarm clock 14, so as to prevent the user from falling asleep.

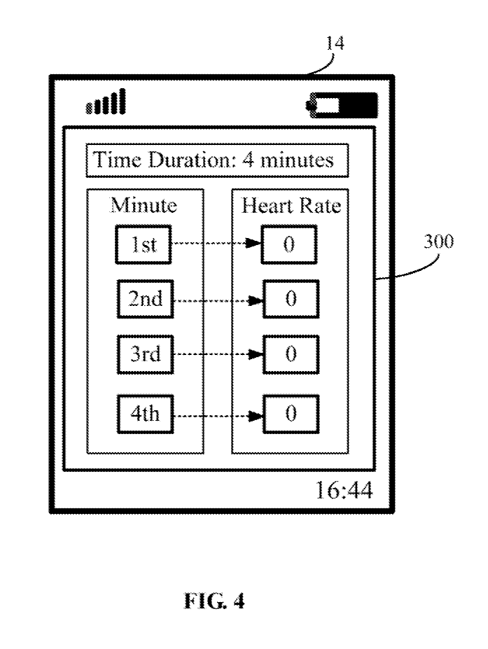

The resetting module 115 resets the heart rate displayed on the display interface 300 to zero, in response to a determination that the plurality of heart rates has not decreased gradually. In one embodiment, as shown in FIG. 4, the resetting module 115 resets the heart rate to zero on the display interface 300 of the display device 14.

FIG. 3 is a flowchart of one embodiment of a method for alarming using the electronic device 1. Depending on the embodiment, additional blocks may be added, while others deleted, and the blocks may also be executed in a different order than described.

In block S300, the setting module 110 sets a time duration in order to measure whether a user wearing the accessory device 2 has fallen asleep, and stores the time duration into the memory system 12 of the electronic device 1. In one embodiment, as shown in FIG. 3, for example, the setting module 110 sets the time duration as 4 minutes.

In block S310, the receiving module 111 receives a plurality of heart rates of the user during the time duration from the accessory device 2 using the BLUETOOTH device 16, and displays the heart rate on the display device 14. In one embodiment, the accessory device 2 periodically (e.g., 1 minute) measures a heart rate of the user using the sensor 20 and sends the heart rate to the electronic device 1 using the BLUETOOTH device 22. For example, the accessory device 2 measures four heart rates 85 beats per minute, 78 beats per minute, 70 beats per minute, and 63 beats per minute within a 4 minutes duration. As shown in FIG. 3, the receiving module 111 receives the four heart rates 85, 78, 70, and 63 from the accessory device 2, then displays the four heart rates 85, 78, 70, and 63 on a display interface 300 of the display device 14.

In block S320, the comparing module 112 compares the plurality of heart rates sequentially. In one embodiment, for example, the comparing module 112 compares the heart rate 85 with the heart rate 78, then compares the heart rate 78 with 70, and compares the heart rate 70 with the heart rate 63.

In block S330, the determining module 113 determines if the plurality of heart rates has decreased gradually according to the comparison. In one embodiment, as shown in FIG. 3, the heart rates are 85, 78, 70, and 63, then the procedure goes to block S340. If not, for example, the heart rates are 85, 78, 70, and 75, block S350 is implemented.

In block S340, the executing module 114 generates an alarm sound to remind the user by the alarm clock 14. In one embodiment, if the plurality of heart rates of the user has decreased gradually, it may indicate that the user has fallen asleep, then the executing module 114 generates an alarm sound to remind the user by the alarm clock 14, so as to prevent the user from falling asleep.

In block S350, the resetting module 115 resets the heart rate displayed on the display interface 300 to zero, in response to a determination that the plurality of heart rates has not decreased gradually, then the procedure goes to block S310. In one embodiment, as shown in FIG. 4, the resetting module 115 resets the heart rate to zero on the display interface 300 of the display device 14.

Although certain inventive embodiments of the present disclosure have been specifically described, the present disclosure is not to be construed as being limited thereto. Various changes or modifications may be made to the present disclosure without departing from the scope and spirit of the present disclosure.

What is claimed is:

1. An electronic device, comprising:
a memory system storing a plurality of programs;
and a processor that executes one or more operations for the plurality of programs, the plurality of programs comprising:
a setting module operable to set a time duration to measure whether a user wearing an accessory device has fallen asleep, and to store the time duration into the memory system of the electronic device, the accessory device measuring a plurality of heart beat rates of the user;
a receiving module operable to receive the plurality of heart rates of the user during the time duration, using a BLUETOOTH device of the electronic device;
a comparing module operable to compare the plurality of heart rates sequentially;
a determining module operable to determine if the plurality of the heart rates has decreased gradually according to the comparison;
an executing module operable to generate an alarm sound to remind the user using an alarm clock of the electronic device, in response to a determination that the plurality of heart rate has decreased gradually; and
a resetting module operable to reset the heart rate to zero, in response to a determination that the plurality of heart rates has not decreased gradually.

2. The electronic device of claim 1, wherein the accessory device comprises a sensor operable to measure the heart rate of the user and a BLUETOOTH device operable to send the heart rate of the user to the electronic device.

3. The electronic device of claim 1, wherein the electronic device is selected from the group consisting of a mobile phone, a personal digital assistant (PDA), a handheld game player, and a digital camera.

4. The electronic device of claim 1, wherein the memory system is selected from the group consisting of a smart media card (SMC), a secure digital card (SDC), a compact flash card (CFC), a multi media card (MMC), a memory system stick (MS), an extreme digital card (XDC), and a trans flash card (TFC).

5. A method for alarming using an electronic device, the method comprising:
setting a time duration to measure whether a user wearing an accessory device has fallen asleep, and to store the time duration into the memory system of the electronic device, the accessory device measuring a plurality of heart beat rates of the user;
receiving the plurality of heart rates of the user during the time duration using a BLUETOOTH device of the electronic device;
comparing the plurality of heart rates sequentially;
determining if the plurality of heart rates has decreased gradually according to the comparison;
generating an alarm sound to remind the user by an alarm clock of the electronic device, in response to a determination that the plurality of heart rates has decreased gradually; and
resetting the plurality of heart rates displayed on a display device to zero, in response to a determination that the plurality of heart rates has not decreased gradually.

6. The method of claim 5, wherein the accessory device comprises a sensor operable to measure the heart rate of the user and a BLUETOOTH device operable to send the heart rate of the user to the electronic device.

7. The method of claim 5, wherein the electronic device is selected from the group consisting of a mobile phone, a personal digital assistant (PDA), a handheld game player, and a digital camera.

8. The method of claim 5, wherein the memory system is selected from the group consisting of a smart media card (SMC), a secure digital card (SDC), a compact flash card (CFC), a multi media card (MMC), a memory system stick (MS), an extreme digital card (XDC), and a trans flash card (TFC).

9. A storage medium having stored thereon instructions that, when executed by an electronic device, causes the electronic device to perform a method for alarming using an electronic device, the method comprising:
setting a time duration to measure whether a user wearing an accessory device has fallen asleep, and to store the time duration into the memory system of the electronic device, the accessory device measuring a plurality of heart beat rates of the user;
receiving the plurality of heart rates of the user during the time duration using a BLUETOOTH device of the electronic device;
comparing the plurality of heart rates sequentially;
determining if the plurality of heart rates has decreased gradually according to the comparison;
generating an alarm sound to remind the user by an alarm clock of the electronic device, in response to a determination that the plurality of heart rates has decreased gradually; and
resetting the plurality of heart rates displayed on a display device to zero, in response to a determination that the plurality of heart rates has not decreased gradually.

10. The medium of claim 9, wherein the accessory device comprises a sensor operable to measure the heart rate of the user and a BLUETOOTH device operable to send the heart rate of the user to the electronic device.

11. The medium of claim 9, wherein the electronic device is selected from the group consisting of a mobile phone, a personal digital assistant (PDA), a handheld game player, and a digital camera.

12. The medium of claim 9, wherein the memory system is selected from the group consisting of a smart media card (SMC), a secure digital card (SDC), a compact flash card (CFC), a multi media card (MMC), a memory system stick (MS), an extreme digital card (XDC), and a trans flash card (TFC).
